(19) United States
(12) Patent Application Publication
(10) Pub. No.: US 2015/0020242 A1
(43) Pub. Date: Jan. 15, 2015

(54) TORENIA-ORIGINATED PROMOTER CAPABLE OF ACTING IN PETALS

(71) Applicant: SUNTORY HOLDINGS LIMITED, Osaka-shi, Osaka (JP)

(72) Inventors: Yoshikazu Tanaka, Mishima-gun (JP); Yukihisa Katsumoto, Mishima-gun (JP)

(73) Assignee: SUNTORY HOLDINGS LIMITED, Osaka-shi, Osaka (JP)

(21) Appl. No.: 14/378,784

(22) PCT Filed: Feb. 19, 2013
(86) PCT No.: PCT/JP2013/054017

§ 371 Date: Aug. 14, 2014

(30) Foreign Application Priority Data
Feb. 24, 2012 (JP) 2012-038644

(51) Int. Cl. C12N 15/82

(52) U.S. Cl. CPC C12N 15/8233; C12N 15/827; C12N 15/825
USPC 800/323; 536/24.1; 435/320.1; 435/419; 435/411; 435/412; 435/415; 435/417

(57) ABSTRACT

A novel promoter useful for altering the color of flowers of a plant, which is selected from the group consisting of: (1) a nucleic acid which comprises the nucleotide sequence represented by SEQ ID NO: 20, SEQ ID NO: 12 or SEQ ID NO: 21; and (2) a nucleic acid which maintains the same promoter activity as that of the nucleotide sequence represented by SEQ ID NO: 20, SEQ ID NO: 12 or SEQ ID NO: 21, and which comprises a nucleotide sequence that is produced by modifying the original nucleotide sequence by the addition, deletion and/or substitution of one or several nucleotide sequences, or can hybridize with a nucleotide acid comprising a nucleotide sequence complementary to the original nucleotide sequence under highly stringent conditions, or has a 90% or more sequence identity to the original nucleotide sequence.

### TECHNICAL FIELD

The present invention relates to a novel promoter. More particularly, the present invention relates to a transcription regulatory region of flavonoid 3′,5′-hydroxylase (abbreviated as F3′5′) gene or flavone synthase (abbreviated as FNS) gene derived from torenia, and to the use thereof.

### BACKGROUND ART

New traits can be imparted to plants by expressing a useful gene in a target plant using genetic recombination technology. Transgenic plants produced in this manner are already grown commercially on a wide scale. Since regulation of gene expression is primarily controlled at the transcription level, transcriptional regulation is of the greatest importance in terms of regulating gene expression. Namely, the transcription of a target gene at a suitable time, in a suitable tissue, and at a suitable strength is important for producing an industrially useful transgenic plant. In many cases, the start of transcription is controlled by a DNA sequence on the 5′-side of a translation region. A region of DNA that determines the starting site of the transcription of a gene and directly regulates the frequency thereof is referred to as a promoter. Promoters are present at a location several by from the 5′-side of a start codon, and frequently include a sequence such as a TATA box. Cis-regulatory elements that bind various transcription regulatory factors are further present on the 5′-side, and the presence of these elements controls such factors as the timing of transcription, the tissue where transcription takes place or the strength of transcription. Transcription regulatory factors are classified into numerous families according to their amino acid sequences. Examples of some more well-known families include Myb-type transcription factors and bHLH (basic helix-loop-helix)-type transcription factors. In actuality, transcription control regions and promoters are frequently used in the same context and are not rigidly distinguished.

Anthocyanins, which are a main component of flower color, are one of the members of secondary metabolites generally referred to as flavonoids. The color of anthocyanins is dependent upon the structure thereof. Namely, flower color becomes blue with an increase in the number of hydroxyl groups on the B ring of anthocyanidins, which are chromophores of anthocyanins. Typical examples of anthocyanidins include delphinidin, cyanidin and pelargonidin, and the number of hydroxyl groups on the B ring of these compounds is 3, 2 and 1, respectively. Among these, delphinidin results in the bluest color (see FIG. 1). In addition, as the number of aromatic acyl groups (such as coumaroyl groups or caffeoyl groups) that modify an anthocyanin increases, the color produced by the anthocyanin is known to become blue (namely, the absorption maximum shifts towards longer wavelengths) and the stability of the anthocyanin is known to increase. Alternatively, in the case a flavonoid such as a flavone is present with an anthocyanin, the resulting color is known to become bluer and deeper. Such effects are referred to as copigment effects, and flavone is a type of copigment (see Non-Patent Document 1).

Extensive research has been conducted on enzymes involved in anthocyanin synthesis and the gene encoding those enzymes (see Non-Patent Document 1). For example, flavonoid 3′-hydroxylase (abbreviated as F3′H) and F3′5′H are enzymes that catalyze a reaction that increases the number of hydroxyl groups on the B ring. F3′H is required for synthesis of cyanidin while F3′5′H is required for the synthesis of delphinidin. Since delphinidin is not synthesized in such plants as roses, carnations and chrysanthemums, there are no blue varieties of these plants. There are examples of changing the flower color of these plants to blue by expressing F3′5′H gene in these plants (see International Publication No. WO 1996/036716, Patent Document 4 and International Publication No. WO 2009/062253). Flavones are synthesized from flavonoids by catalysis by the enzyme FNS. There are examples of changing the flower color of petunia by expressing flavone synthase gene therein (see Plant Biotechnol., 21, 377-386 (2004)).

Moreover, findings have also been obtained regarding the transcriptional regulation of genes involved in the biosynthesis of anthocyanins. A cis-regulatory element is present in the transcription control region of these genes located on the 5′-side of a start codon that binds with an Myb-type transcription regulatory factor and bHLH-type transcription regulatory factor. Myb-type transcription regulatory factor and bHLH-type transcription regulatory factor are known to control the synthesis of anthocyanins in plants such as petunia, corn and perilla (see Non-Patent Document 1).

In plants, promoters responsible for gene transcription (to also be referred to as transcription regulatory regions) consist of so-called constitutional promoters that function in any tissue and at any time in the developmental stage, organ/tissue-specific promoters that function only in specific organs and tissues, and time-specific promoters that are expressed only at specific times in the developmental stage. Constitutional promoters are frequently used as promoters for expressing useful genes in transgenic plants. Typical examples of constitutional promoters include cauliflower 35S promoter and promoters constructed on the basis thereof (see Non-Patent Document 3) and Mac1 promoter (see Non-Patent Document 4). In plants, however, many genes are expressed organ/tissue-specifically or time-specifically. This indicates that it is necessary for plants that these genes be expressed in specific tissues or organs or at specific times. There are examples of carrying out gene recombination in plants using such tissue/organ-specific or time-specific transcription regulatory regions. For example, protein has been made to accumulate in seeds by using a seed-specific transcription regulatory region.

There are several reports on promoters that function specifically in flower petals or primarily function in flower petals along with their use. For example, there are examples of changing flower color by expressing F3′5′H gene in petunia or carnation using the promoter of chalcone synthase gene derived from snapdragon or petunia. The promoter of rose chalcone synthase gene has been shown to function in rose and chrysanthemum. There are also examples of changing flower color by expressing the promoter of cineraria F3′5′H gene in petunia. In addition, there are also examples of using the flavanone 3-hydroxylase gene promoter of chrysanthemum to express F3′5′H gene in chrysanthemum petals.

However, it is difficult to predict the degree to which a promoter will function and bring about a desired phenotype in a target recombinant plant. In addition, gene silencing may occur if a base sequence identical or similar to a host plant is introduced or if numerous copies of an introduced gene are inserted or that gene is inserted repeatedly (see Non-Patent Document 5). Thus, the repeated use of the same promoter to express a plurality of exogenous genes should be avoided since it can cause gene silencing. In addition, even though the enzyme genes involved in flavonoid synthesis may function in flower petals, the timing of their expression differs. In general, flavones and flavonols are known to be expressed when flowers are younger in comparison with anthocyanins. For example, the acquisition of a plurality of promoters having different expression timing is industrially important for changing flower color.

### PRIOR ART DOCUMENTS

### Patent Documents

Patent Document 1: International Publication No. WO96/25500
Patent Document 2: International Publication No. WO01/72984
Patent Document 3: International Publication No. WO94/28140
Patent Document 4: International Publication No. WO05/17147

### Non-Patent Documents

Non-Patent Document 1: Plant J., 54, 737-749, 2008
Non-Patent Document 2: Agricultural and Biological Chemistry, 53, 797-800, 1989
Non-Patent Document 3: Plant Cell Physiology, 1996, 37, 49-59
Non-Patent Document 4: Plant Molecular Biology, 1990, 15, 373-381
Non-Patent Document 5: Annals of Botany, 1997, 79, 3-12

### DISCLOSURE OF THE INVENTION

### Problems to be Solved by the Invention

An object of the present invention is to provide a novel promoter that is useful for changing the flower color of a plant.

### Means for Solving the Problems

As a result of conducting extensive studies to solve the aforementioned problems, the inventors of the present invention discovered that the transcription regulatory regions of torenia-derived F3′5′H gene an FNS gene are novel promoters that are useful for changing the flower color of a plant and confirmed the usefulness thereof, thereby leading to completion of the present invention.

Namely, the present invention is as described below.

[1] A nucleic acid selected from the group consisting of the following:

(1) a nucleic acid composed of the base sequence shown in SEQ ID NO: 20;

(2) a nucleic acid able to function as a transcription regulatory region of torenia F3′5′H gene and composed of a base sequence that has been modified by addition, deletion and/or substitution of one or several base sequences in the base sequence shown in SEQ ID NO: 20;

(3) a nucleic acid able to function as a transcription regulatory region of torenia F3′5′H gene and hybridize under highly stringent conditions with a nucleic acid composed of a base sequence complementary to the base sequence shown in SEQ ID NO: 20; and,

(4) a nucleic acid able to function as a transcription regulatory region of torenia F3′5′H gene and having at least 90% sequence identity with the base sequence shown in SEQ ID NO: 20.

[2] An expression vector containing the nucleic acid described in [1] above.

[3] The expression vector described in [2] above, containing the base sequence shown in SEQ ID NO: 20.

[4] A non-human host transformed by the expression vector described in [2] or [3] above.

[5] A plant, or a progeny, a portion or a tissue thereof, introduced with the nucleic acid described in [1] above.

[6] The plant, or progeny, portion or tissue thereof, described in [5] above, which is a cut flower.

[7] A processed cut flower obtained by using the cut flower described in [6] above as a raw material.

[8] A nucleic acid selected from the group consisting of the following:

(1) a nucleic acid composed of the base sequence shown in SEQ ID NO: 12;

(2) a nucleic acid able to function as a transcription regulatory region of torenia F3′5′H gene and composed of a base sequence that has been modified by addition, deletion and/or substitution of one or several base sequences in the base sequence shown in SEQ ID NO: 12;

(3) a nucleic acid able to function as a transcription regulatory region of torenia F3′5′H gene and hybridize under highly stringent conditions with a nucleic acid composed of a base sequence complementary to the base sequence shown in SEQ ID NO: 12; and,

(4) a nucleic acid able to function as a transcription regulatory region of torenia F3′5′H gene and having at least 90% sequence identity with the base sequence shown in SEQ ID NO: 12.

[9] An expression vector containing the nucleic acid described in [8] above.

[10] The expression vector described in [9] above, containing the base sequence shown in SEQ ID NO: 12.

[11] A non-human host transformed by the expression vector described in [9] or [10] above.

[12] A plant, or a progeny, a portion or a tissue thereof, introduced with the nucleic acid described in [8] above.

[13] The plant, or progeny, portion or tissue thereof, described in [12] above, which is a cut flower.

[14] A processed cut flower obtained by using the cut flower described in [13] above as a raw material.

[15] A nucleic acid selected from the group consisting of the following:

(1) a nucleic acid composed of the base sequence shown in SEQ ID NO: 21;

(2) a nucleic acid able to function as a transcription regulatory region of torenia F3′5′H gene and composed of a base sequence that has been modified by addition, deletion and/or substitution of one or several base sequences in the base sequence shown in SEQ ID NO: 21;

(3) a nucleic acid able to function as a transcription regulatory region of torenia FNS gene and hybridize under highly stringent conditions with a nucleic acid composed of a base sequence complementary to the base sequence shown in SEQ ID NO: 21; and,

(4) a nucleic acid able to function as a transcription regulatory region of torenia FNS gene and having at least 90% sequence identity with the base sequence shown in SEQ ID NO: 21.

[16] An expression vector containing the nucleic acid described in [15] above.

[17] The expression vector described in [16] above, containing the base sequence shown in SEQ ID NO: 21.

[18] A non-human host transformed by the expression vector described in [16] or [17] above.

[19] A plant, or a progeny, a portion or a tissue thereof, introduced with the nucleic acid described in [15] above.

[20] The plant, or progeny, portion or tissue thereof, described in [19] above, which is a cut flower.

[21] A processed cut flower obtained by using the cut flower described in [20] above as a raw material.

[22] An expression vector containing the nucleic acid described in [22] above and the nucleic acid described in [15] above.

[23] The expression vector described in [22] above, containing the base sequence shown in SEQ ID NO: 20 and the base sequence shown in SEQ ID NO: 21.

[24] A non-human host transformed by the expression vector described in [22] or [23] above.

[25] A plant, or a progeny, a portion or a tissue thereof, introduced with the nucleic acid described in [1] above or the nucleic acid described in [15] above.

[26] The plant, or progeny, portion or tissue thereof, described in [25] above, which is a cut flower.

[27] An expression vector containing the nucleic acid described in [8] above and the nucleic acid described in [15] above.

[28] The expression vector described in [27] above, containing the base sequence shown in SEQ ID NO: 12 and the base sequence shown in SEQ ID NO: 21.

[29] A non-human host transformed by the expression vector described in [27] or [28] above.

[30] A plant, or a progeny, a portion or a tissue thereof, introduced with the nucleic acid described in [8] above or the nucleic acid described in [15] above.

[31] The plant, or progeny, portion or tissue thereof, described in [30] above, which is a cut flower.

[32] A processed cut flower obtained by using the cut flower described in [31] above as a raw material.

### Effects of the Invention

The use of promoter regions thought to govern the transcription of enzyme genes in torenia flower petals, namely regions that control the transcription of torenia F3′5′H gene and FNS gene, makes it possible to specifically induce transcription of an exogenous gene in tissue such as flower tissue in which flavonoids and anthocyanins accumulate. Examples of transcribed exogenous genes include, but are not limited to, genes involved in flower color, scent and the like.

### BRIEF DESCRIPTION OF THE DRAWINGS

FIG. 1 is a schematic diagram of the biosynthesis pathway of flavonoids.

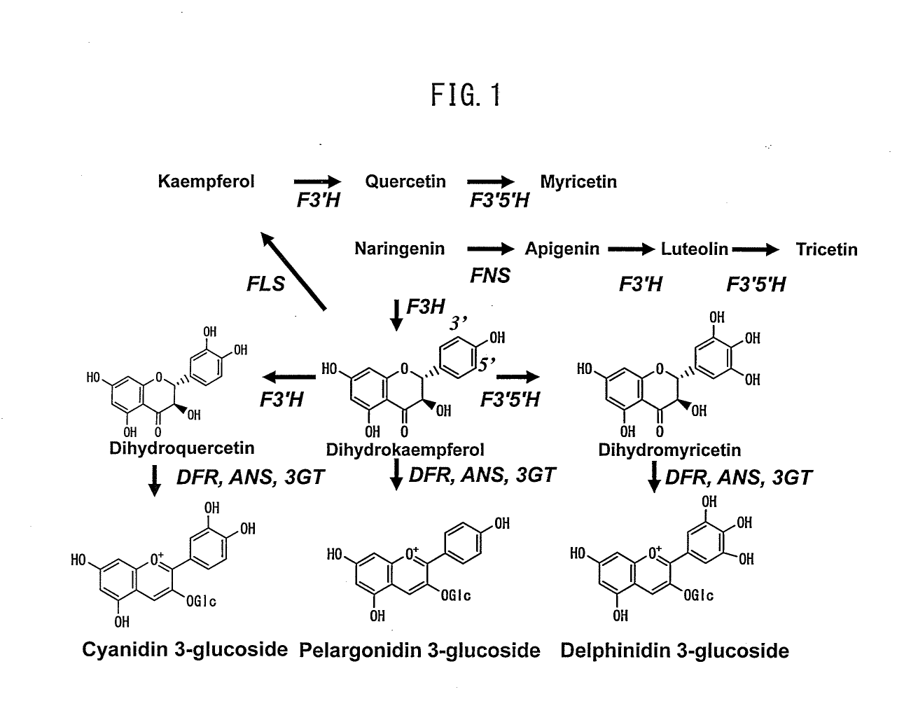

FIG. 2 is a schematic diagram of a binary vector pSPB3797 for gene insertion.

FIG. 3 is a schematic diagram of a binary vector pSPB3798 for gene insertion.

### BEST MODE FOR CARRYING OUT THE INVENTION

Examples of transcription regulatory regions of the present invention include nucleic acids composed of the base sequences shown in SEQ ID NO: 20, SEQ ID NO: 12 and SEQ ID NO: 21. However, promoters composed of base sequences that have been modified by addition, deletion and/or substitution of one or more (1, 2, 3, 4, 5, 6, 7, 8, 9 or 10) bases in the base sequence shown in SEQ ID NO: 20, SEQ ID NO: 12 or SEQ ID NO: 21 are also thought to maintain activity that is the same as that of the original promoter. Thus, the present invention also relates to a nucleic acid composed of a base sequence that has been modified by addition, deletion and/or substitution of one or several base sequences in the base sequence shown in SEQ ID NO: 20, SEQ ID NO: 12 or SEQ ID NO: 21 provided the nucleic acid functions as a transcription regulatory region in flower petals.

The present invention also relates to a nucleic acid that is able to function as a transcription regulatory region of torenia F3′5′H gene or FNS gene and hybridize under highly stringent conditions with the base sequence shown in SEQ ID NO: 20, SEQ ID NO: 12 or SEQ ID NO: 21, as well as a nucleic acid that is able to function as a transcription regulatory region of torenia F3′5′H gene or FNS gene and has at least 90% sequence identity with the base sequence shown in SEQ ID NO: 20, SEQ ID NO: 12 or SEQ ID NO: 21.

Examples of these nucleic acids include nucleic acids composed of a base sequence capable of hybridizing under stringent conditions with a polynucleotide complementary to the base sequence shown in SEQ ID NO: 20, SEQ ID NO: 12 or SEQ ID NO: 21 and having sequence identity with the base sequence shown in SEQ ID NO: 20, SEQ ID NO: 12 or SEQ ID NO: 21 of preferably about 70% or more, more preferably about 80%, 81%, 82%, 83%, 84%, 85%, 86%, 87%, 88%, 89%, 90%, 91%, 92%, 93%, 94%, 95%, 96%, 97% or 98%, and most preferably about 99%.

Here, the term “stringent conditions” refers to hybridization conditions easily determined by a person with ordinary skill in the art, and are typically experimental conditions dependent upon probe length, washing temperature and salt concentration. In general, the temperature for proper annealing becomes higher the longer the probe, while the temperature becomes lower the shorter the probe. Hybridization is generally dependent on the ability of a complementary strand to reanneal denatured DNA in the case of being present in an environment at a temperature close to but lower than the melting point thereof. More specifically, an example of lowly stringent conditions consists of washing in a 5×SSC and 0.1% SDS solution under temperature conditions of 37° C. to 42° C. in the stage of washing the filter following hybridization. In addition, an example of highly stringent conditions consists of washing in a 0.1×SSC and 0.1% SDS solution at 65° C. Increasing the stringency of these conditions allows the obtaining of a polynucleotide having higher sequence homology or sequence identity.

The present invention also relates to a vector containing the transcription regulatory region of torenia F3′5′H gene and/or FNS gene, and a non-human host transformed by that vector.

Moreover, the present invention also relates to a plant, or a progeny, portion or tissue thereof, having a useful trait such as change of color obtained by linking the transcription regulatory region of torenia F3′5′H gene and/or FNS gene to a useful exogenous gene and introducing that exogenous gene therein, and the portion may be a cut flower. Examples of plants able to be transformed include, but are not limited to, rose, chrysanthemum, carnation, snapdragon, cyclamen, orchid, dahlia, bluebell, freesia, Transvaal daisy, gladiola, baby's breath, kalanchoe, lily, pelargonium, geranium, petunia, torenia, tulip, rice, morning glory, barley, wheat, rape, potato, tomato, poplar, banana, eucalyptus, sweet potato, soybean, alfalfa, lupine and corn.

The present invention also relates to a processed product using the aforementioned cut flower (processed cut flower). Here, examples of processed cut flowers include, but are not limited to, pressed flowers, preserved flowers, dry flowers and resin-sealed flowers using these cut flowers.

### EXAMPLES

The following provides a detailed explanation of the invention according to examples thereof.

Molecular biological techniques were in accordance with Molecular Cloning (Sambrook and Russell, 2001) unless specifically indicated otherwise. Although methods used to transform Agrobacterium, petunia and rose are described in International Publication No. WO2004/020637 or Patent Document 4, the methods are not limited thereto.

### Example 1

### Cloning of Transcription Control Region of Torenia F3′5′H Gene TBG10

The base sequence of torenia F3′5′H cDNA is known (Molecular Breeding 6, 239-246 (2000), Gene Bank DNA Accession No. AB012925). A chromosomal DNA library of torenia was prepared using λDASHII (Agilent Technologies, Inc.) for the vector in accordance with the method recommended by the manufacturer. Torenia chromosomal DNA was prepared from the leaves of the torenia variety Summer Wave Blue (Suntory Flowers Ltd.). The resulting torenia chromosomal DNA library was screened using labeled torenia F3′5′H cDNA and the plaque of a phage that hybridized with torenia F3′5′H cDNA was recovered. Using this phage as a template, PCR was carried out using two types of oligonucleotides (T3pro: 5′-AATTAACCCTCACTAAAGGG-3′ (SEQ ID NO: 1), T7pro: 5′-TAATACGACTCACTATAGGG-3′ (SEQ ID NO: 2)) as primers to amplify a DNA fragment containing the sequence derived from torenia chromosomal DNA. Using this DNA as a template, PCR was carried out using one set of oligonucleotides (T3pro, THF2RV: 5′-CTATGGAAGATAACAATG-3′ (SEQ ID NO: 3)) or one set of oligonucleotides (T7pro, THF2RV) as primers. An approximately 5.6 kb fragment (SEQ ID NO: 4) able to be amplified by PCR using T3pro and THF2RV was cloned in pCR2.1 TOPO (Invitrogen Corp.). The resulting plasmid was designated as pSPB3745.

Using the aforementioned phage as a template, PCR was carried out using one set of oligonucleotides (TBG10proFW: 5′-TGAAATATAAATATGAATGGG-3′ (SEQ ID NO: 5), TBG10proRV: 5′-ACTGAATGGTGACTAGCTGC-3′ (SEQ ID NO: 6)) as primers. The resulting DNA fragment was cloned in BluntII-TOPO (Invitrogen Corp.) to obtain plasmid pSPB3764 (containing SEQ ID NO: 7). This DNA sequence is shown in SEQ ID NO: 7. Moreover, using pSPB3764 as a template, PCR was carried out using one set of oligonucleotides (TBG10proFW, TBG10proXbaIRV: 5′-TCTAGACTGAATGGTGACTAGC-3′ (SEQ ID NO: 8)) as primers. This DNA fragment was cloned in BluntII-TOPO to obtain plasmid pSPB3770. This plasmid contains in an approximately 4 kb 5′-untranslated region of torenia F3′5′H and has a restrictase XbaI recognition sequence on the 3′-terminal thereof.

### Example 2

### Cloning of Transcription Control Region of Torenia F3′5′H Gene TBG16

Plaque of the phage that hybridized with torenia F3′5′H cDNA obtained in Example 1 was allowed to proliferate to obtain phage DNA. Using this as a template, PCR was carried out using one set of oligonucleotides (T3pro, THF2RV) or one set of oligonucleotides (T7pro, THF2RV) as primers. An approximately 2.3 kb DNA fragment obtained from the (T3pro, THF2RV)-derived PCR product was cloned in pCR2.1 TOPO to obtain plasmid pSPB3746. The sequence thereof is shown in SEQ ID NO: 9. Using this plasmid as a template, PCR was carried out using a set of oligonucleotides (TBG16proFW: 5′-TCCTATTGCACTCGTTTTTTC-3′ (SEQ ID NO: 10), TBG16proRV: 5′-ACTGAATGGTGACTAGCCGĈ3′ (SEQ ID NO: 11)) as primers. The resulting DNA fragment was cloned in BluntII-TOPO (Invitrogen Corp.) to obtain plasmid pSPB3758. The DNA sequence contained in this plasmid is shown in SEQ ID NO: 12. Moreover, using pSPB3758 as a template, PCR was carried out using one set of oligonucleotides (TBG16proFW, TBG16proBamHI: 5′-GGATCCACTGAATGGTGACTAGCC-3′ (SEQ ID NO: 13)) as primers. This DNA fragment was cloned in BluntII-TOPO to obtain plasmid pSPB3768. This plasmid contains in an approximately 0.7 kb 5′-untranslated region of torenia F3′5′H and has a restrictase BamHI recognition sequence on the 3′-terminal thereof.

### Example 3

### Cloning of Transcription Control Region of Torenia FNS Gene

The torenia chromosomal DNA library obtained in Example 1 was screened using labeled torenia FNS cDNA, and plaque of a phage hybridized with torenia FNS cDNA was recovered. The phage plaque was allowed to proliferate to prepare phage DNA. Using this DNA as a template, PCR was carried out using one set of oligonucleotides (T3pro, TFNSR3: 5′-ATTCCTAATGGGCTGAAAGTG-3′ (SEQ ID NO: 14)) or one set of oligonucleotides (T7pro, TFNSR3) as primers. An approximately 4.2 kb DNA fragment (SEQ ID NO: 15) able to be amplified by PCR using T7pro and TFNSR3 was cloned in pCR2.1 TOPO. The resulting plasmid was designated as pSPB3747.

Using the aforementioned phage DNA as a template, PCR was carried out using one set of oligonucleotides (TFNS1proFW: 5′-CAAATGAAACCCCATCAGTGTC-3′ (SEQ ID NO: 16), TFNS1proRV: 5′-GCTTTATATATATTTTTTTAGCGC-3′ (SEQ ID NO: 17)) as primers. The amplified DNA fragment was cloned in BluntII-TOPO to obtain plasmid PSPB3759. The sequence inserted with this plasmid is shown in SEQ ID NO: 18. Using pSPB3759 as a template, PCR was carried out using one set of oligonucleotides (TNFS1proFW, TFNS1pBamHIRV: 5′-GGATCCGCTTTATATATATTTTTTTAGC-3′ (SEQ ID NO: 19) as primers. This DNA fragment was cloned in BluntTOPO to obtain plasmid pSPB3769. This plasmid contains an approximately 3.6 kb 5′-untranslated region of torenia FNS and has a restrictase BamHI recognition sequence on the 3′-terminal thereof.

### Example 4

### Production of Binary Vector Containing Torenia F3′5′H and FNS Transcription Control Regions

The plasmid pSPB3770 obtained in Example 1 was digested and blunted with SspI and further digested with XbaI. The resulting 1.6 kb DNA fragment (SEQ ID NO: 20) was linked to a DNA fragment obtained by digesting and blunting pBinPLUS with HindIII and further digesting with XbaI to obtain plasmid pSPB3791.

A DNA fragment obtained by digesting plasmid pSPB580 (described in Patent Document 4) with BamHI and Pad (containing pansy-derived F3′H′SH BP40 cDNA and a petunia D8 terminator sequence) was linked to pBinPLUS obtained by digesting with BamHI and Pad to obtain plasmid pSPB3795. A DNA fragment obtained by digesting plasmid pSPB3791 with XbaI and Pad was linked to a DNA fragment obtained by digesting pSPB3795 with XbaI and Pad to obtain binary vector pSPB3793. A TBG10-derived transcription control region, pansy-derived F3′5′H cDNA and the petunia D8 terminator were linked in this binary vector in that order.

The plasmid pSPB3768 obtained in Example 2 was digested and blunted with EcoRI and further digested with BamHI. The resulting approximately 0.7 kb DNA fragment was linked to a DNA fragment obtained by digesting and blunting pBinPLUS with HindIII and further digesting with BamHI to obtain plasmid pSPB3790. A DNA fragment obtained by digesting this with BamHI and PacI was then linked to a DNA fragment obtained by digesting pSPB580 with BamHI and Pad to obtain binary vector pSPB3792. A TBG16-derived transcription control sequence, pansy-derived F3′5′H cDNA and the petunia D8 terminator were linked in this binary vector in that order.

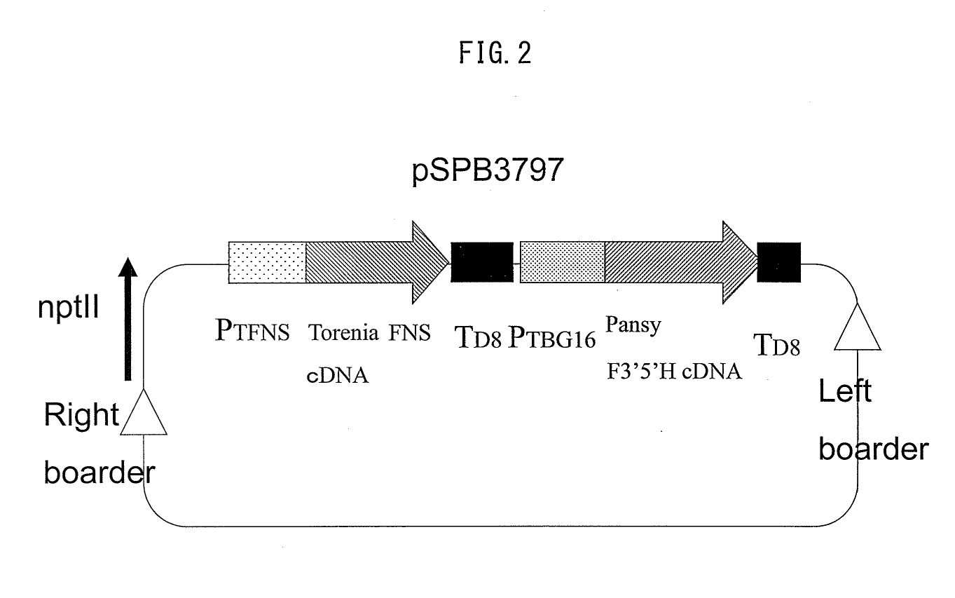

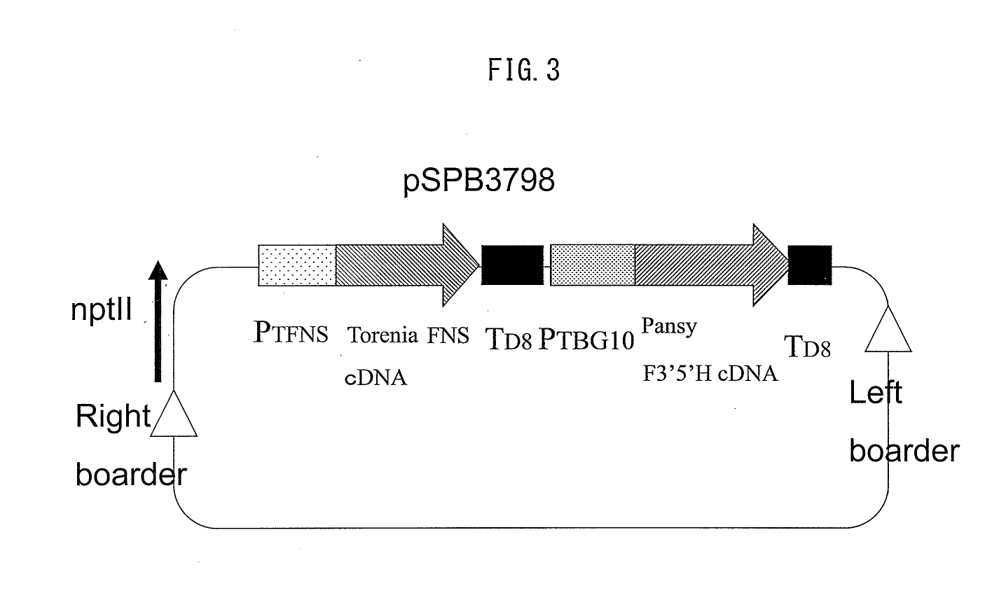

The plasmid pSPB3769 obtained in Example 3 was digested and blunted with SpeI and further digested with BamHI. The resulting approximately 1.4 kb DNA fragment (SEQ ID NO: 21) was linked to a DNA fragment obtained by digesting and blunting pSPB3383 with HindIII and further digesting with BamHI to obtain plasmid pSPB3789. A torenia FNS transcription control region, torenia FNS cDNA and petunia D8 terminator were linked in this plasmid in that order, and a DNA fragment in which they were contained was able to be recovered by cleaving with AscI. The binary vector introduced with this DNA fragment at the AscI site of pSPB3793 was designated as pSPB3798 (see FIG. 3), while the binary vector introduced with this DNA fragment at the AscI site of pSPB3792 was designated as pSPB3797 (see FIG. 2).

### Example 5

### Expression in Petunia (Results for PT315)

Plasmid pSPB3797 or pSPB3798 described in Example 4 was introduced into Agrobacterium tumefaciens. Petunia strain Skr4xSw63 (described in International Publication No. WO93/01290) that produces light pink-colored flowers was transformed using this transformed Agrobacterium. An experimental plot derived from pSPB3797 was designated as PT314 and an experimental plot derived from pSPB3798 was designated at PT315. Two independent strains of transformants were obtained in PT314. The flower color of both strains changed to light reddish-violet. The results of analyzing the flower petals of PT314 and PT315 and the anthocyanidins of Skr4xSw63 using known methods are shown in the following Tables 1 and 2.

TABLE 1

| | Del- | | | | |
|---|---|---|---|---|---|
| Sample | phinidin | Petunidin | Pelargonidin | Peonidin | Malvidin |
| PT314-1 | 5.4 | 98.6 | 3.8 | 20.6 | 1950 |
| PT314-2 | 4.2 | 76.5 | 3.3 | 17.6 | 1150 |
| Skr4xSw63 | 0 | 2.9 | 22.5 | 15.8 | 60.3 |

Units: μg/g fresh flower petals

Twenty-six independent strains of transformants were obtained in PT315. Among these, the flower color of 15 strains changed to reddish-violet. The flower petals of a portion of these strains and the anthocyanidins and flavones of Skr4xSw63 were analyzed using known methods.

TABLE 2

| Sample | Delphinidin | Petunidin | Pelargonidin | Peonidin | Malvidin | Apigenin | Tricetin |
|---|---|---|---|---|---|---|---|
| PT315-1 | 3.0 | 72 | 0 | 2.7 | 1330 | 2.8 | 11 |
| PT315-9 | 6.8 | 126 | 0 | 0.6 | 1580 | 1.9 | 31 |
| PT315-12 | 2.0 | 33 | 1.0 | 7.0 | 393 | 44 | 9.4 |
| PT315-13 | 3.0 | 72 | 0 | 2.8 | 1550 | 4.9 | 17 |
| PT315-14 | 4.0 | 72 | 0 | 5.3 | 1290 | 5.6 | 11 |
| PT315-15 | 5.0 | 109 | 0 | 1.0 | 1630 | 4.6 | 30 |
| PT315-17 | 5.6 | 116 | 0 | 8.2 | 1690 | 18 | 27 |
| PT315-19 | 1.9 | 39 | 1.5 | 11 | 756 | 3.0 | 7.0 |
| PT315-20 | 3.6 | 93 | 0 | 4.8 | 1190 | 3.3 | 17 |
| PT315-21 | 10 | 185 | 0 | 14 | 1130 | 1.4 | 8.1 |
| PT315-25 | 5.8 | 101 | 0 | 0.6 | 1540 | 2.4 | 11 |
| PT315-26 | 3.3 | 71 | 0 | 1 | 862 | 3.5 | 25 |
| Skr4xSw63 | 0 | 3.2 | 5.6 | 11 | 74 | 0 | 0 |

Units: μg/g fresh flower petals

The above results indicate that a TBG10-derived transcription control region contained in pSPB3770, a TBG16-derived transcription control region contained in pSPB3768, and an FNS-derived transcription control region contained in pSPB3759 demonstrate their functions in different strains of petunia as well.

### Example 6

### Introduction of pSPB3798 into Rose Variety “Ocean Song”

Binary plasmid pSPB3798 described in Example 4 was introduced into Agrobacterium tumefaciens strain Ag10. Rose variety “Ocean Song” was transformed using this transformed Agrobacterium to obtain 15 transformants. Among the 15 transformants subjected to anthocyanidin analysis, accumulation of delphinidin was confirmed in 5 transformants. The maximum delphinidin content was 7.3% (mean: 5.0%). In addition, as a result of analyzing flavonols and flavones, myricetin was detected in 8 of 9 transformants and tricetin was detected in 7 transformants. Delphinidin, myricetin and tricetin were all formed by the action of pansy-derived F3′5′H, and the transcription control region of torenia-derived TBG10 gene was indicated to function in rose.

On the other hand, since previously absent accumulation of flavones (tricetin, luteolin, apigenin) was able to be confirmed in all the transformants subjected to analysis, the transcription control region of torenia-derived FNS gene was also indicated to function in rose. Furthermore, the total amount of flavones was a maximum of 0.6588 mg per 1 g of fresh flower petals.

The analysis values of representative transformants are shown in the following Table 3.

TABLE 3

| Plant | Del | Anthocyanidins (mg/g) | | | Flavonols (mg/g) | | | Flavones (mg/g) | | | |
|---|---|---|---|---|---|---|---|---|---|---|---|
| No. | (%) | Del | Cya | Pel | M | Q | K | Tri | Lut | Api | Total |
| Ocean | | | | | | | | | | | |
| Song | | | | | | | | | | | |
| Control | 0.05 | 0.0000 | 0.0324 | 0.0000 | 0.0000 | 2.6657 | 0.1730 | 0.0000 | 0.0000 | 0.0000 | 0.0000 |
| 1 | 7.3% | 0.0006 | 0.0076 | 0.0000 | | | | | | | |
| 2 | 6.0% | 0.0006 | 0.0100 | 0.0000 | 0.0662 | 1.3835 | 0.2738 | 0.0431 | 0.2928 | 0.0138 | 0.3498 |
| 3 | 5.4% | 0.0002 | 0.0036 | 0.0000 | | | | | | | |
| 4 | 4.8% | 0.0004 | 0.0090 | 0.0000 | | | | | | | |
| 5 | 1.4% | 0.0002 | 0.0141 | 0.0000 | 0.0109 | 1.7564 | 0.2911 | 0.0110 | 0.1680 | 0.0088 | 0.1878 |
| 6 | 0.0% | 0.0000 | 0.0019 | 0.0000 | 0.0041 | 0.5825 | 0.2806 | 0.0112 | 0.3098 | 0.0206 | 0.3417 |
| 7 | 0.0% | 0.0000 | 0.0026 | 0.0000 | 0.0000 | 0.05060 | 0.3112 | 0.0063 | 0.3153 | 0.0222 | 0.3439 |
| 8 | 0.0% | 0.0000 | 0.0106 | 0.0000 | 0.0075 | 1.4460 | 0.4400 | 0.0056 | 0.0825 | 0.0059 | 0.0941 |
| 9 | 0.0% | 0.0000 | 0.0037 | 0.0000 | 0.0137 | 0.8253 | 0.2856 | 0.0577 | 0.5679 | 0.332 | 0.6588 |
| 10 | 0.0% | 0.0000 | 0.0492 | 0.0000 | 0.0341 | 3.5195 | 0.4933 | 0.0000 | 0.1222 | 0.0066 | 0.1289 |

Del: delphinidin, Cya: cyanidin, Pel: pelargonidin, M: myricetin, Q: quercetin, K: kaempferol, Tri: tricetin, Lut: luteolin, Api: apigenin Del (%): Percentage of delphinidin among total anthocyanidins

### Example 7

### Introduction of pSPB3797 into Rose Variety “Ocean Song”

Binary vector pSPB3797 described in Example 4 was introduced into the lavender-colored rose variety “Ocean Song” and 7 transformants were obtained. Accumulation of delphinidin was unable to be confirmed in any of the 7 transformants analyzed for anthocyanidins. In addition, as a result of analyzing for flavonols and flavones, there were no transformants in which myricetin or tricetin was detected. These results indicate that the promoter of torenia-derived TBG16 gene does not function in rose.

On the other hand, since accumulation of flavones (luteolin, apigenin) due to the action of torenia-derived FNS was able to be confirmed in all analyzed transformants, the promoter of torenia-derived FNS gene was indicated to function in rose. Furthermore, the total amount of flavones was a maximum of 0.1704 mg per 1 g of fresh flower petals.

Analysis values of representative transformants are shown in the following Table 4.

TABLE 4

| Plant | Del | Anthocyanidins (mg/g) | | | Flavonols (mg/g) | | | Flavones (mg/g) | | | |
|---|---|---|---|---|---|---|---|---|---|---|---|
| No. | (%) | Del | Cya | Pel | M | Q | K | Tri | Lut | Api | Total |
| Ocean | | | | | | | | | | | |
| Song | | | | | | | | | | | |
| Control | 0.05 | 0.0000 | 0.0324 | 0.0000 | 0.0000 | 2.6657 | 0.1730 | 0.0000 | 0.0000 | 0.0000 | 0.0000 |
| 1 | 0.0% | 0.0000 | 0.0123 | 0.0000 | 0.0000 | 0.1610 | 0.2288 | 0.0000 | 0.1405 | 0.0076 | 0.1481 |
| 2 | 0.0% | 0.0000 | 0.0034 | 0.0000 | 0.0000 | 0.8202 | 0.2264 | 0.0000 | 0.1367 | 0.0082 | 0.1449 |
| 3 | 0.0% | 0.0000 | 0.0106 | 0.0000 | 0.0000 | 1.2625 | 0.5670 | 0.0000 | 0.1292 | 0.0102 | 0.1394 |
| 4 | 0.0% | 0.0000 | 0.0190 | 0.0000 | 0.0000 | 1.9873 | 0.9176 | 0.0000 | 0.0702 | 0.0048 | 0.0750 |
| 5 | 0.0% | 0.0000 | 0.0077 | 0.0000 | 0.0000 | 1.6323 | 0.4398 | 0.0000 | 0.1651 | 0.0053 | 0.1704 |
| 6 | 0.0% | 0.0000 | 0.0208 | 0.0000 | 0.0000 | 2.4281 | 0.6282 | 0.0000 | 0.1067 | 0.0052 | 0.1119 |
| 7 | 0.0% | 0.0000 | 0.0154 | 0.0000 | 0.0000 | 2.1013 | 0.3322 | 0.0000 | 0.1528 | 0.0062 | 0.1590 |

Del: delphinidin, Cya: cyanidin, Pel: pelargonidin, M: myricetin, Q: quercetin, K: kaempferol, Tri: tricetin, Lut: luteolin, Api: apigenin Del (%): Percentage of delphinidin among total anthocyanidins

### INDUSTRIAL APPLICABILITY

Promoter regions thought to govern transcription of enzyme genes in torenia flowers, namely the transcription regulatory regions of torenia flavonoid 3′,5′-hydroxlyase gene and flavone synthase gene, were determined to function as transcription regulatory regions in the flower petals of different species of flowers in the form of petunia and rose. Thus, it is possible to specifically induce transcription of an exogenous gene in tissue such as flower tissue in which anthocyanins accumulate by using these transcription regulatory regions. Examples of transcribed exogenous genes include, but are not limited to, genes involved in flower color, scent and the like.

1. A nucleic acid selected from the group consisting of the following:
(1) a nucleic acid composed of the base sequence shown in SEQ ID NO: 20;
(2) a nucleic acid able to function as a transcription regulatory region of torenia F3′5′H gene and composed of a base sequence that has been modified by addition, deletion and/or substitution of one or several base sequences in the base sequence shown in SEQ ID NO: 20;
(3) a nucleic acid able to function as a transcription regulatory region of torenia F3′5′H gene and hybridize under highly stringent conditions with a nucleic acid composed of a base sequence complementary to the base sequence shown in SEQ ID NO: 20; and,
(4) a nucleic acid able to function as a transcription regulatory region of torenia F3′5′H gene and having at least 90% sequence identity with the base sequence shown in SEQ ID NO: 20.

2. An expression vector containing the nucleic acid according to claim 1.

3. The expression vector according to claim 2, containing the base sequence shown in SEQ ID NO: 20.

4. A non-human host transformed by the expression vector according to claim 2.

5. A plant, or a progeny, a portion or a tissue thereof, introduced with the nucleic acid according to claim 1.

6. The plant, or progeny, portion or tissue thereof, according to claim 5, which is a cut flower.

7. A processed cut flower obtained by using the cut flower according to claim 6 as a raw material.

8. A nucleic acid selected from the group consisting of the following:
(1) a nucleic acid composed of the base sequence shown in SEQ ID NO: 12;
(2) a nucleic acid able to function as a transcription regulatory region of torenia F3′5′H gene and composed of a base sequence that has been modified by addition, deletion and/or substitution of one or several base sequences in the base sequence shown in SEQ ID NO: 12;
(3) a nucleic acid able to function as a transcription regulatory region of torenia F3′5′H gene and hybridize under highly stringent conditions with a nucleic acid composed of a base sequence complementary to the base sequence shown in SEQ ID NO: 12; and,
(4) a nucleic acid able to function as a transcription regulatory region of torenia F3′5′H gene and having at least 90% sequence identity with the base sequence shown in SEQ ID NO: 12.

9. An expression vector containing the nucleic acid according to claim 8.

10. The expression vector according to claim 9, containing the base sequence shown in SEQ ID NO: 12.

11. A non-human host transformed by the expression vector according to claim 9.

12. A plant, or a progeny, a portion or a tissue thereof, introduced with the nucleic acid according to claim 8.

13. The plant, or progeny, portion or tissue thereof, according to claim 12, which is a cut flower.

14. A processed cut flower obtained by using the cut flower according to claim 13 as a raw material.

15. A nucleic acid selected from the group consisting of the following:
(1) a nucleic acid composed of the base sequence shown in SEQ ID NO: 21;
(2) a nucleic acid able to function as a transcription regulatory region of torenia F3′5′H gene and composed of a base sequence that has been modified by addition, deletion and/or substitution of one or several base sequences in the base sequence shown in SEQ ID NO: 21;
(3) a nucleic acid able to function as a transcription regulatory region of torenia FNS gene and hybridize under highly stringent conditions with a nucleic acid composed of a base sequence complementary to the base sequence shown in SEQ ID NO: 21; and,
(4) a nucleic acid able to function as a transcription regulatory region of torenia FNS gene and having at least 90% sequence identity with the base sequence shown in SEQ ID NO: 21.

16. An expression vector containing the nucleic acid according to claim 15.

17. The expression vector according to claim 16, containing the base sequence shown in SEQ ID NO: 21.

18. A non-human host transformed by the expression vector according to claim 16.

19. A plant, or a progeny, a portion or a tissue thereof, introduced with the nucleic acid according to claim 15.

20. The plant, or progeny, portion or tissue thereof, according to claim 19, which is a cut flower.

21. A processed cut flower obtained by using the cut flower according to claim 19 as a raw material.

22. An expression vector containing the nucleic acid according to claim 1.

23. The expression vector according to claim 22, containing the base sequence shown in SEQ ID NO: 20 and the base sequence shown in SEQ ID NO: 21.

24. A non-human host transformed by the expression vector according to claim 22.

25. A plant, or a progeny, a portion or a tissue thereof, introduced with the nucleic acid according to claim 1.

26. The plant, or progeny, portion or tissue thereof, according to claim 25, which is a cut flower.

27. An expression vector containing the nucleic acid according to claim 8.

28. The expression vector according to claim 27, containing the base sequence shown in SEQ ID NO: 12 and the base sequence shown in SEQ ID NO: 21.

29. A non-human host transformed by the expression vector according to claim 27.

30. A plant, or a progeny, a portion or a tissue thereof, introduced with the nucleic acid according to claim 8.

31. The plant, or progeny, portion or tissue thereof, according to claim 30, which is a cut flower.

32. A processed cut flower obtained by using the cut flower according to claim 31 as a raw material.


SEQUENCE LISTING (21 sequences)

SEQ ID NO: 1 - DNA, 20 bp, Artificial Sequence
  primer T3pro
aattaaccctcactaaaggg

SEQ ID NO: 2 - DNA, 20 bp, Artificial Sequence
  primer T7pro
taatacgactcactataggg

SEQ ID NO: 3 - DNA, 18 bp, Artificial Sequence
  primer THF2RV
ctatggaagataacaatg

SEQ ID NO: 4 - DNA, 5606 bp, Torenia Fournieri
  misc_feature: Trenia F3′5′H TBG10 region in pSPB3745
aattaaccctcactaaagggaacgaattcggatcctcatgttatgaaatataaatatgaatgggattaaacttgtagttaattggtatcattaaaaaaaaaagaaaaaagaaattgaagtaaaataatcgcaggtaacttgaagaaaatattaataactaaaataataataataataataataataataagcaggtttgaaacacacaccttgggaatttgttgttcttgacagccttttgaccatatttcctccatcgatatccatcgtcaagtatatcgacttggcttcttgtctgaaaggcgtatttatgcttcttattcttcttcttcttattcttctcctcgctctttattgcttccttattattatcagacttgtttgggtcctcctctgcgagagttattgcagtactattattattaaagctactactggcgattgtaatagcatctgacatgcttttaatattcatctccatgctgttatagccaaaactactactgctaggcctgtgatcttgaaacagatcataatgatcttgtggaaatgaagaagaagactaaaacattattggatagttattattgtccatgagaagagaagatgataatagataacaaataaatgatggttttccttgtttttttttataataactatgaatgtgaagaatagtggattaagaaaagtacaagtcgtcgtgtggtgttgtttagaatgagagaaaagtttattatacaacaagtaccgtcccagctcgtttaaagatgaaaggcggcggcttgacttgagggcgaatattattatttatttaaataagttacatcataaacatattttgtgcatagtgaatgttaaaccaaaagcgaaatggctatgatcgcagtattaatatattatctgaggagataaatttgatcgaattgattagcagggataataattatagagatgatccaacttgcttgatgaaattatggatctatactcaatcttaaaacgaatttacttacttacatagcgtgaaatagctaattggaaatgaaaagaaaaagaaaaagaaaaattgttagaaatatgacttggagggagggttttgaatcttcaaacatacacaaatttaattaatacattacggtcgttaagcaaatcatttttattctacggacagtagatattgcaataatagttagaaaagttggtaaagaattagtacagttggtcaaacactattattattattattattattgttattattattattattctataaatctgataataaaaacaaatagcatatgaataatagcatataaatgctggatccgatttaattttaattttagttaaatagcaactttcagtaaatttttttagttaactgcctctggaaaaatgaaatgcgtgggtgggagcggttcggatttggtacggaaaatgcttttctccgcattgtgcggataaatctggaccgtgcgatcaggcccaaaattaatccagtggtgaaggacgagagggggtaaatctggaatttttttttttactcataataaaaaacgattatatcttttgaatgccaaaacaaaataaaaaaataaaaacaccaaatttcatcatttttaattccctacaaaaagagatccatcttgctatataaattttatatttttagggtttagggtttagggtttacggcctggcacagacacatctaaaacgaagcacaactcagctaaacgaagcacaaattcattattatgaagcacatattcattagtatgaagcacatattcatttttatgaagcgcaattcatttgtatgaagcacatattcatttttatgaagcgcaattcatttgtatgaagcacaaaatcatttgtatgaaacttataatcatttgtatgaagcacaaattcatttatatgaagcaaaaattcatttgtatgaacgcaattcatttgtatgaatcacaaattaatttgtatgaagcacaaattaatttgtatgaagcacaactcatttgtacaaaatcatacaaataattataagtaccatctaattgaagcacaaattcatttgtttgtatgaagcacaaattcatttgtataaagcacaaattcatttgtacaatacatccaaataatatataaagcaccatctaaacgaagtacaaattcatttgtatgaagcacaaattcgtacaaaaaatatttaaagcacaatctaaaacgaagcacagattcatttgtatgaagcacaaattcatttgtacaatacatccaaataatatataaagcaccatctaaacgaagcacaaattcatttgtatgaagcacaaattcatttgtacaaattcatacaaaaaatatttaaagcacaatctaaaacgaagcacagattcatttgtatgaagcacagattcatcaggggtattttagacaaaaacccgacgaatatcatccgttagatttattttgagtaaatccaatggcccattacttttgtccgcacatacggagaaaagtaccgtccgcatttgaaccagaggggatgggagggactgcgaacataatataattcgggtcgatggcgtaccaaaaaatggtcaaataaataatgctacaagttaaatgcacgtgattctcttgttgaccgttaaatgttgatttctattttgatcgaattttatagatattagctcagctgctgagactgagagagagattattttagaaacggatctgatcaagatcgctcgtgattattctcatcatctcgtgtagcatttagatttggtaaaaccgtaaaaatatcttgtcttaacttcttcctattaattccacaatctcacgcaaaaatgtatttttacagcgacatgccttgaggaggaatttcagtgctaaagacaaaatagacttagaaatgtgccgtttgattaattttggtagttaaaaaaaaaaaattatttatttatttatgcttagccataataattaggacggaaacaagaagaatttgcccatttttaaggcctacgaatagtatacgggccataactttatgggggacattttcaggcctacccaatatgattgaatgtgcctccactccaattatcaaaaagaaacgccgactgagcttgagcacgattttcaaatgtctactccacagtgcaaaaattgcacgatcaattctgatgaaatgtattcccatctatgtagatgaatgggagcgtttgggatccaacactcgttttttctacctatttcgacggttttctctggtcaggtggtttccgagaggggactgaaatatacagtacagttgttgagatattgatgagatcaatcaagtcgtgtaagcttatctttctgaaaatataattatatttaattgattatcctaccgcttgtcaattttttctattgcgagtgtgttttggtgtaaagagtagctatcgatgctgaagagatttttatcaagtaagatgatgtggagaactaactgattgtttgttttgttgccagcgatatattgatgatgatgttctttgcaattagagttattctacttaacaaactttttctcttgtcataacatacacatagatactgcatgcttttccatgattcattctatctaataaaggtcaatctactccccactttcatccatgccaatgaaacacatacataaatggaaacatatcataatttaggtgtggccctattagttttcttctcgttgaacgtcggttgatggcctaaaaaaacgtgacaaaccacacagttgtcacccaacgtactccattgatacacatataaagctcagtgattgattattcataaacactcaactggttgagcactatttgcctcttctttgctagattcacatcgagtaagtactccgtccgaccttttattgtatggtataatcgtaagaaaacaaattaagcagctagtcaccattcagtatgacagtagctactacacttgtcgttgtgttgagtagagagttgttgtatgtgttggttgtctacattctggtctcaaaatcactatctaccatcatcgtcgtctcccgcaaacggttaccccccggcccaacgggcttccccgtggtcggtgccctcccattgttgggcagcatgcctcacgtggcgttggccaaaatggcaaaaaaatacgggcccgtcatgtaccttaaaacgggcacactgggcatggtggtggcctccacgccttcctcggcaagagcgtttctcaagacactggactccaacttctcgaaccggcccgccaatgccggtgccacccacctggcctacggtgcgcaggacatggtgttcgctccctacggccccagatggagactgctgagaaggttaagcaacctccacatgctcggcggcaaggccctcgacggatgggcaaacgtcagggcctcggagttggggcacatgctggaggccatgaaccgggcgagccgggagggggaggtggtggtggtgccggagatgctggtgtacgccatggccaacatgatcgggcaagtcacactgagccggcgggtcttcgtgaccaaaggggaggaagtgaacgagtttaaggacatggtggtggagctcatgacgtctgctggatacttcaacatcggggactttgtcccgatccttgcttggatggacttgcaagggattgagagagggatgaaagctcttcacaaaaagtttgacaagctcatcacgagaatgttggagcagcatgaggcgtcggcccacaagcccgattttctcgacgccatcatcgcggccagccttgacgattattcgagtgaggaggagaggctcagtacgtccaacatcaaagctctcttattggtaaggctaagctttattaattttacccatcattgaatatgcatgcaacaacaagaggtcctatcattgaatatgcatgtgtgtgtgtagaacttgtttactgctggaacggacacatcatcgagcacgatagaatgggcactctcggagatgctgaaaaaggggaaaatcctaaagagggctcaagaggagatggacagagtggtgggacgagagaggcgcttggtggaatcggacatagagaaactggggtacctgaaagccatatgcaaagagacgttccggaagcatccttcgacgccgctgaacctgcctcggatctcgtcggaggcatgcgtggtgaacggccactacataccaaagggcacgaggctgagcgtcaacatatgggccatcgggagagaccctgaggtgtgggaggatccgttggagtttaacccggacaggtttctgcactcgaagatggatccgagggggaacgatttcgagctcatgcccttcggtgcgggtcgaaggatatgtgccgggacgaggatggggatagtcctcgtcgagtatatattggccactttggtgcattctttcggctggaaagcggctgatcaagataacaacatcatgaatatggaggaggcctttgggattgctctgcagaaggccacacctcttaaggctctcgttactcctcgcctgcctttacattgttatcttccatag

SEQ ID NO: 5 - DNA, 21 bp, Artificial Sequence
  primer TBG10proFW
tgaaatataaatatgaatggg

SEQ ID NO: 6 - DNA, 20 bp, Artificial Sequence
  primer TBG10proRV
actgaatggtgactagctgc

SEQ ID NO: 7 - DNA, 3942 bp, Torenia Fournieri
  misc_feature: Trenia F3′5′H TBG10  in pSPB3764
tgaaatataaatatgaatgggattaaacttgtagttaattggtatcattaaaaaaaaaagaaaaaagaaattgaagtaaaataatcgtaggtaacttgaagaaaataataataactaaaataataataataataataataataataagcaggtttgaaacacacaccttgggaatttattgttcttgacagccttttgaccatatttcctccatcgatatccatcgtcaagtatatcgacttggcttcttgtctgaaaggcgtatttatgcttcttattcttcttcttcttattcttctcctcgctctttattgcttccttattattatcagacttgtttgggtcctcctctgcgagagttattgcagtactattattattaaagctactactggcgattgtaatagcatctgacatgcttttaatattcatctccatgctgttatagccaaaactactactgctaggcttgtgatcttgaaacagatcataatgatcttgtggaaatgaagaagaagactaaaacattattggatagttattattgtccatgagaagagaagatgataatagataacaaataaatgatggttttccttgttttttttttataataactatgaatgtgaagaatagtggattaagaaaagtacaagtcgtcgtgtggtgttgtttagaatgagagaaaagtttattatacaacaagtaccgtcccagctcgtttaaagatgaaaggcggcggcttgacttgagggcgaatattattatttatttaaataagttacatcataaacatatttttgtgcatagtgaatgttaaaccaaaagcgaaatggctatgatcgcagtattaatatattatctgaggagataaatttgatcgaattgattagcagggataataattatagagatgatccaacttgcttgatgaaattatggatctatactcaatcttaaaacgaatttacttacttacatagcgtgaaatagctaattggaaatgaaaagaaaaagaaaaagaaaaattgttagaaatatgacttggagggagggttttgaatcttcaaacatacacaaatttaattaatacattacggtcgttaagcaaatcatttttattctacggacagtagatattgcaataatagttagaaaagttggtaaagaattagtacagttggtcaaacactattattattattattattattgttattattattattattctataaatctgataataaaaacaaatagcatatgaataatagcatataaatgctggatccgatttaattttaattttagttaaatagcaactttcagtaaatttttttagttaactgcctctggaaaaatgaaatgcgtgggtgggagcggttcggatttggtacggaaaatgcttttctccgcattgtgcggataaatctggaccgtgcgatcaggcccaaaattaatccagtggtgaaggacgagagggggtaaatctggaattttttttttactcataataaaaaacgattatatcttttgaatgccaaaacaaaataaaaaaataaaaacaccaaatttcatcatttttaattccctacaaaaagagatccatcttgctatataaattttatatttttagggtttagggtttagggtttacggcctggcacagacacatctaaaacgaagcacaactcagctaaacgaagcacaaattcattattatgaagcacatattcattagtatgaagcacatattcatttttatgaagcgcaattcatttgtatgaagcacatattcatttttatgaagcgcaattcatttgtatgaagcacaaaatcatttgtatgaaacttataatcatttgtatgaagcacaaattcatttatatgaagcaaaaattcatttgtatgaacgcaattcatttgtatgaatcacaaattaatttgtatgaagcacaaattaatttgtatgaagcacaactcatttgtacaaaatcatacaaataattataagtaccatctaattgaagcacaaattcatttgtttgtatgaagcacaaattcatttgtataaagcacaaattcatttgtacaatacatccaaataatatataaagcaccatctaaacgaagtacaaattcatttgtatgaagcacaaattcgtacaaaaaatatttaaagcacaatctaaaacgaagcacagattcatttgtatgaagcacaaattcatttgtacaatacatccaaataatatataaagcaccatctaaacgaagcacaaattcatttgtatgaagcacaaattcatttgtacaaattcatacaaaaaatatttaaagcacaatctaaaacgaagcacagattcatttgtatgaagcacagattcatcaggggtattttagacaaaaacccgacgaatatcatccgttagatttattttgagcaaatccaatggcccattacttttgtccgcacatacggagaaaagtaccgtccgcatttgaaccagaggggatgggagggactgcgaacataatataattcgggtcaatggcgtaccaaaaaatggtcaaataaataatgctacaagttaaatgcacgtgattctcttgttgaccgttaaatgttgatttctattttgatcgaattttatagatattagctcagctgctgagactgagagagagattattttagaaacggatctgatcaagatcgctcgtgattattctcatcatctcgtgtagcatttagatttggtaaaaccgtaaaaatatcttgtcttaacttcttcctattaattccacaatctcacgcaaaaatgtatttttacagcgacatgccttgaggaggaatttcagtgctaaagacaaaatagacttagaaatgtgccgtttgattaattttggtagttaaaaaaaaaaaaattatttatttatttatgcttagccataataattaggacggaaacaagaagaatttgcccatttttaaggcctacgaatagtatacgggccataactttatgggggacattttcaggcctacccaatatgattgaatgtgcctccactccaattatcaaaaagaaacgccgactgagcttgagcacgattttcaaatgtctactccacagtgcaaaaattgcacgatcaattctgatgaaatgtattcccatctatgtagatgaatgggagcgtttgggatccaacactcgttttttctacctatttcgacggttttctctggtcaggtggtttccgagaggggactgaaatatacagtacagttgttgagatattgatgagatcaatcaagtcgtgtaagcttatctttctgaaaatataattatatttaattgattatcctaccgcttgtcaattttttctattgcgagtgtgttttggtgtaaagagtagctatcgatgctgaagagatttttatcaagtaagatgatgtggagaactaactgattgtttgttttgttgccagcgatatattgatgatgatgttctttgcaattagagttattctacttaacaaactttttctcttgtcataacatacacatagatactgcatgcttttccatgattcattctatctaataaaggtcaatctactccccactttcatccatgccaatgaaacacatacataaatggaaacatatcataatttaggtgtggccctattagttttcttctcgttgaacgtcggttgatggcctaaaaaaacgtgacaaaccacacagttgtcacccaacgtactccattgatacacatataaagctcagtgattgattattcataaacactcaactggttgagcactatttgcctcttctttgctagattcacatcgagtaagtactccgtccgaccttttattgtatggtataatcgtaagaaaacaaattaagcagctagtcaccattcagt

SEQ ID NO: 8 - DNA, 22 bp, Artificial Sequence
  primer TBG10proXbaIRV
tctagactgaatggtgactagc

SEQ ID NO: 9 - DNA, 2307 bp, Torenia Fournieri
  misc_feature: Trenia F3′5′H TBG16 region in pSPB3746
aattaaccctcactaaagggaacgaattcggatcctattgcactcgttttttctcgacgattttctctggtcaggtccgttgtttggattccgacaggagactgaaatatacagttgttgagctgctgatgagatcaatcaagtcatctaagcttatctttctgaaatatataattatatttaattgattatcgtaccgcttgtcaattttttgtctattgcgagtgcgttctggtggaattggatgtggccgcgatgcctggtgtagtgtagctgaagagattttcgagtgattgctggttttgttgccagcgatattgatgttctttgcagttaagagttattctacttaccaatttttctcataacgtacacaaaaaaccattctatctatctaataaaggtcaatctactcaccactttcatccatgccataaatggaaacatatcataagttaggtgtgtatcgttggataattttgccctattacttttttttttcctcgttgaacgtcggttgatggccttaaaaaacgtgacaaaccacacagttgtcacccaactccattgatacacatataaagctcagtaattgattatctcaactagctgagcattatttgccctttctttgctagattcacattgtgtaagtactcggaccttttgttgtataatcagaagaaaacaaattaagcggctagtcaccattcagtatgacagtagctactacacttgtcgttgtgttgagtagagagttgttgtatgtgttggttgtctacgttctggtgcggtcaatactcatctcaaagtcactgtctaccatcatcgtctcccgcaaacggttaccccccggcccaacgggcttccccgtggtcggtgccctcccattgttgggcaacatgcctcacgtggcgttggccagaatggcaaaaaaatacgggcccgtcatgtaccttaaaacgggcacactggggatggtggtggcctccacgccttcgtcggcaaaagcgttcctcaagacactggactccaacttctcgaaccgccctggcaatgccggtgccacccacctggcctacggggcgcaggacatggtgtttgctccctacggccccagatggagactgctgagaaggttaagcaatctccacatgctcggaggcaaggccgtggacggatgggcaaacgtcagggccttgagttggggcacatgctggaggccatgaaccgggcgagccgcgacggggaggtggtggtggtgccggagatgctggtgtacgccatggccaacatgatcgggcaagtgatactgagccggcgggttttcgtcaccaaaggggatgaagtgaacgagtttaaggacatggtggtggagctcatgacgactgctggatacttcaacatgggcgactttgtcccgatccttgcttggatggacttgcaagggattgagagagggatgaaagcccttcacaaaaagttggacaagctcatcacgagaatgttggagcagcatgcggcgtcggcccacacgcccgattttctcgacgccatcagccttgacgactattcgagtggggagaggctcagtacgaccaacatcaaagctctcttattggtaagctttatatattttccccatgccatgccatccatcctattgaaatatgtgtataagtggtgtagaacttgtttactgcgggaacggacacatcatcgagcacgatagaatgggcgctctcggagatgctgaaaaaccggaaaatcctaaggaggaaatggacagagtggtgggacgagagaggcggctggtggaatcggacatagaaaaactgccgtacctggtagccatatgcaaggagactttccggaagcatccttcgacgccgctgaaccggcctcggatgtcgtcggaggcgtgcgtggtgaacggtcactacatacccaagggcacgaggctgagcgtcaacatatgggccatcgggagagaccctgaggtgtgggaggatccgttggaatttaacccggacaggtttctgcactcgaagatggatccgagggggaacgatttcgagctcatgcccttcggtgcgggtcgaaggatatgtgccgggacgaggatggggattgtcctcgtcgagtacatattgggcactttggtgcattctttccaatggaagctgccgcctcatcaagacaccatcaatatggaggaggcctttgggattgctctgcagaaggccatacataccacttgaggctctcgttactcctcgccttcctttacattgttatcttccatag

SEQ ID NO: 10 - DNA, 21 bp, Artificial Sequence
  primer TBG16proFW
tcctattgcactcgttttttc

SEQ ID NO: 11 - DNA, 20 bp, Artificial Sequence
  primer TBG16proRV
actgaatggtgactagccgc

SEQ ID NO: 12 - DNA, 685 bp, Torenia Fournieri
  misc_feature: Trenia 3′5′H TBG16 region in pSPB3758
tcctattgcactcgttttttctcgacgattttctctggtcaggtccgttgtttggattccgacaggagactgaaatatacagttgttgagctgctgatgagatcaatcaagtcatctaagcttatctttctgaaatatataattatatttaattgattatcgtaccgcttgtcaattttttgtctattgcgagtgcgttctggtggaattggatgtggccgcgatgcctggtgtagtgtagctgaagagattttcgagtgattgctggttttgttgccagcgatattgatgttctttgcagttaagagttattctacttaccaatttttctcataacgtacacaaaaaaccattctatctatctaataaaggtcaatctactcaccactttcatccatgccataaatggaaacatatcataagttaggtgtgtatcgttggataattttgccctattactttttttttttcctcgttgaacgtcggttgatggccttaaaaaacgtgacaaaccacacagttgtcacccaactccattgatacacatataaagctcagtaattgattatctcaactagctgagcattatttgccccttctttgctagattcacattgtgtaagtactcggaccttttgttgtataatcagaagaaaacaaattaagcggctagtcaccattcagt

SEQ ID NO: 13 - DNA, 24 bp, Artificial Sequence
  primer TBG16proBamHI
ggatccactgaatggtgactagcc

SEQ ID NO: 14 - DNA, 21 bp, Artificial Sequence
  primer TFNSR3
attcctaatgggctgaaagtg

SEQ ID NO: 15 - DNA, 4173 bp, Torenia Fournieri
  misc_feature: Trenia FNS transcriptional region in pSPB3747
tccttggcaattttatttttattttgtttaatgaagaagcgaagtacagttccttggatcctgcaaatgaaaccccatcagtgtccacaaatcatcatatatcttgtccttcaatttctataccctacaaaggttagtccttcagtctttctcaagcaaaagtcattttccaatggcttagaaagaaaaagacataacttttgtatgtcatcaatacattagctactgtaggcattttgacctcttcaatattagaaggaaaataaaaaaagaaacaaagcacagagaccattaaagctacgtacatctccagcttagcttggcaataatatttctaaaataatttctttctccccatatttcatcgtagcagaggccttaatttcaagaattcttacaagggtttctacttattgtaatatcatagggtttatttggtatttctttccttcaagctgtttcacatcaacatattcaagatgtttcccaaaaacacatacctgattcctcagatttctcctattcattctcgcgccactacttcttcagttcttgacctgaacgaccccgagatcttgtattgtcaccaccaccaagacatgttctctgctcacaattatatatcagcttcaaataatgctcaggctctcgagccttcgatgatgttcaatcaagatatctcaggcctaaacattggagatccgaagatgtcgcccgttaaacactctgaatgaactctcactagaacatccaatccattcccgatatctccctgcacaaatacgtacacgaaaggttagtcgagatacagcatagcatttttgaggttgaattattatttttaccttcacgagagctatatgttccatctcgtcgatcttcgatcggtagctatgagcctgaaacaccccccatttcgttggcagacgagtaattgctgttctctccaccagcttttcccttttcctcatgtacctgcggatagatacttgaataatattatcgttgtcactcgttctttgcagtctctatatgttgtgtcgaggctttgagctttacctaatgagatctgtgatcgaaacgatagggatactatgatccgagcccaattttctcaaatgggccatcgaggccatagaaccatcttctctatcgacgatagtcgagaggacagagacaggtctcaaaccagcttgcatcaccaagtccacagaggcctcggtgtgaccagcccgacgaagaacacctccgtttctatacttgagcgggaacacgtggcccggcttgttgaaatcttcgggcttcgactcgagagaagacagagcaagtacagttttagccctgtccgaagctgacacgccagtagatgtttcagctttagcgtcctaaattgtttaaagagattattggtgagacaaaagaaaaggttaatgctttcgggacatgacacatctaagatcgatctacacgggcaataagcgtagacaacgggtaatatgattaccactgtgatagtgaacgaaggagcagaagaatcatgttctttctcgggattcatcaatggcagcttcaatctctcgaggtcgtctcctttcatcccgacagagacgatgcccgatccgtgtttgatcataaaggcgatggccttctcgctcgcgtgagaagctgccattatgagatttccttctaggctatcgccgctctcatcatctacgacaatcacaaacttcaaacacgaaaaggaaatttcagagacagacgctatgggttttgaaattgcgtcgcaaaatcaaaacagagcctgtgttagcagtacctttccctgtcgcagagcagatagagcattttcaatggatgagtaaccctgagatggacgatcaggatcgccttcggcgtcactcacgaagaaatcaacggtttcgtgagtgatctctgcttcgagcgtttcaaagggtgtagcgggagatgacgactcgtcatcaaatagggagccatttaaggttgcccgtttcttgattgaactctcatcgtcgtcagcatgagataagtctcctgctgaaaatttaagagcatgacagtagtaagtgttgagcttgcatggtctgcttctgatgattccgaatcccaggttggaatgcgaagattttgacgttataaacaatagcgattgctgaagacagataatgcaatccattgattcttgaattgttttatttctatttggatagtaaatgaagagctttgggttatatccagtagatgcagagatttggaaccacatgaatgtaggaatagagacaacaaggccaagaatcccaactagtagataatatcgtgaatgatatttgtaaagaacgttgtattaaattcaggacacttaattgatgaatcacaatcaaataatattattttgcagtatctggtacataagtatgatataaatgataagtgccataactgtgagaatataaactataatcgtaataaaaatttgtatagcataaaatatttaatatttactataaattcaataaaatcaatgctacatgattatgtaagatgatgtggattttcttaacaaaatgattttggcttgatcgtgatgatactattaaaaaataatgaagtttgtctaggacatggcattggtttatagatagatgtcctcatacgtttattggcacttgacatttgtttctttttattcttgtgaattgggatatgtggatttataaatatttttagaagtctggcttgtatttctgatactaatttttatgatataatttctttatgaattaattgatattaaaggctcatttgaagtttgcggatgattcaatgcgagcaatagcgtatatattgtgattggttggtgcactgcacatataggctcgcatcgaatcatccgccgtttgcttaggaaattataagactcattttattaatttaaaaatagaatttgggtgaaaggtagacgtacggtacggctacggtacctaaccttcgaggcttagactaagggtaaagaaaagaccaggaaaagaaaaaatctagaatcgttaaaatattaaatgcaattaaatgaaaataaaaaaggataaataaagatccacaccacacctatcaattgtggagtctaattgtttgaacttgatacaatacaatgatatctgaagtatcgtatcgaaatacacacgttatgtttttcctatttggcacatcatatttattcttggtttttcaaatccaagatacaaaaactcataaaatacctcagtcaccaaaaataacactaacagatttcaaacatactgcatgattatttaaaattaaaaccgagaagtgagatgtccaagaattgtttgttaaaaccctcaaaactctaaggatattacctaataagtcgttggatgtaattcttatgatgatcacaaatcacacattcaatagttcaacgcaattaccgaaagtagcagggcgaaaatgtcattatcatgatcaaagggaccatgaatgtaattgcgcaatcagacgacgtgtcggtgaaaccaaaaaatttagaagaaaatatagctcactcaccaccactactcgaacctaacaccgcattaaatatcctacaccacttcctttctataaagaacacatcaacccacaaagcttttatctcgtttacatcgaaaccgctatatcattacatttacaacagcgctaaaaaaatatatataaagcatggacacagtcttaatcacactctacaccgccctgttcgtcatcaccaccaccttcctcctcctcctccgccgaaggggaccaccgtctccgcccggtcctctctccctacccataattggccacctccacctcctcggcccaagactccaccacacgttccatgaattctcactcaaatacggcccattgatccagctcaagctcggctcgatcccgtgcgtcgtggcctcgacgcccgagctcgcgagagagtttcttaagacgaacgagctcgcgttctcctctcgcaagcactctacggccatagacatcgtcacctacgactcgtcctttgctttctctccgtacggaccctactggaagtacatcaagaaactgtgtacctacgagctgctcggagcgaggaacctcggacactttcagcccattaggaat

SEQ ID NO: 16 - DNA, 22 bp, Artificial Sequence
  primer TFNS1proFW
caaatgaaaccccatcagtgtc

SEQ ID NO: 17 - DNA, 24 bp, Artificial Sequence
  primer TFNS1proRV
gctttatatatatttttttagcgc

SEQ ID NO: 18 - DNA, 3735 bp, Torenia Fournieri
  misc_feature: FNS transcriptional region in pSPB3759
tccttggcaattttatttttattttgtttaatgaagaagcgaagtacagttccttggatcctgcaaatgaaaccccatcagtgtccacaaatcatcatatatcttgtccttcaatttctataccctacaaaggttagtccttcagtctttctcaagcaaaagtcattttccaatggcttagaaagaaaaagacataacttttgtatgtcatcaatacattagctactgtaggcattttgacctcttcaatattagaaggaaaataaaaaaagaaacaaagcacagagaccattaaagctacgtacatctccagcttagcttggcaataatatttctaaaataatttctttctccccatatttcatcgtagcagaggccttaatttcaagaattcttacaagggtttctacttattgtaatatcatagggtttatttggtatttctttccttcaagctgtttcacatcaacatattcaagatgtttcccaaaaacacatacctgattcctcagatttctcctattcattctcgcgccactacttcttcagttcttgacctgaacgaccccgagatcttgtattgtcaccaccaccaagacatgttctctgctcacaattatatatcagcttcaaataatgctcaggctctcgagccttcgatgatgttcaatcaagatatctcaggcctaaacattggagatccgaagatgtcgcccgttaaacactctgaatgaactctcactagaacatccaatccattcccgatatctccctgcacaaatacgtacacgaaaggttagtcgagatacagcatagcatttttgaggttgaattattatttttaccttcacgagagctatatgttccatctcgtcgatcttcgatcggtagctatgagcctgaaacaccccccatttcgttggcagacgagtaattgctgttctctccaccagcttttcccttttcctcatgtacctgcggatagatacttgaataatattatcgttgtcactcgttctttgcagtctctatatgttgtgtcgaggctttgagctttacctaatgagatctgtgatcgaaacgatagggatactatgatccgagcccaattttctcaaatgggccatcgaggccatagaaccatcttctctatcgacgatagtcgagaggacagagacaggtctcaaaccagcttgcatcaccaagtccacagaggcctcggtgtgaccagcccgacgaagaacacctccgtttctatacttgagcgggaacacgtggcccggcttgttgaaatcttcgggcttcgactcgagagaagacagagcaagtacagttttagccctgtccgaagctgacacgccagtagatgtttcagctttagcgtcctaaattgtttaaagagattattggtgagacaaaagaaaaggttaatgctttcgggacatgacacatctaagatcgatctacacgggcaataagcgtagacaacgggtaatatgattaccactgtgatagtgaacgaaggagcagaagaatcatgttctttctcgggattcatcaatggcagcttcaatctctcgaggtcgtctcctttcatcccgacagagacgatgcccgatccgtgtttgatcataaaggcgatggccttctcgctcgcgtgagaagctgccattatgagatttccttctaggctatcgccgctctcatcatctacgacaatcacaaacttcaaacacgaaaaggaaatttcagagacagacgctatgggttttgaaattgcgtcgcaaaatcaaaacagagcctgtgttagcagtacctttccctgtcgcagagcagatagagcattttcaatggatgagtaaccctgagatggacgatcaggatcgccttcggcgtcactcacgaagaaatcaacggtttcgtgagtgatctctgcttcgagcgtttcaaagggtgtagcgggagatgacgactcgtcatcaaatagggagccatttaaggttgcccgtttcttgattgaactctcatcgtcgtcagcatgagataagtctcctgctgaaaatttaagagcatgacagtagtaagtgttgagcttgcatggtctgcttctgatgattccgaatcccaggttggaatgcgaagattttgacgttataaacaatagcgattgctgaagacagataatgcaatccattgattcttgaattgttttatttctatttggatagtaaatgaagagctttgggttatatccagtagatgcagagatttggaaccacatgaatgtaggaatagagacaacaaggccaagaatcccaactagtagataatatcgtgaatgatatttgtaaagaacgttgtattaaattcaggacacttaattgatgaatcacaatcaaataatattattttgcagtatctggtacataagtatgatataaatgataagtgccataactgtgagaatataaactataatcgtaataaaaatttgtatagcataaaatatttaatatttactataaattcaataaaatcaatgctacatgattatgtaagatgatgtggattttcttaacaaaatgattttggcttgatcgtgatgatactattaaaaaataatgaagtttgtctaggacatggcattggtttatagatagatgtcctcatacgtttattggcacttgacatttgtttctttttattcttgtgaattgggatatgtggatttataaatatttttagaagtctggcttgtatttctgatactaatttttatgatataatttctttatgaattaattgatattaaaggctcatttgaagtttgcggatgattcaatgcgagcaatagcgtatatattgtgattggttggtgcactgcacatataggctcgcatcgaatcatccgccgtttgcttaggaaattataagactcattttattaatttaaaaatagaatttgggtgaaaggtagacgtacggtacggctacggtacctaaccttcgaggcttagactaagggtaaagaaaagaccaggaaaagaaaaaatctagaatcgttaaaatattaaatgcaattaaatgaaaataaaaaaggataaataaagatccacaccacacctatcaattgtggagtctaattgtttgaacttgatacaatacaatgatatctgaagtatcgtatcgaaatacacacgttatgtttttcctatttggcacatcatatttattcttggtttttcaaatccaagatacaaaaactcataaaatacctcagtcaccaaaaataacactaacagatttcaaacatactgcatgattatttaaaattaaaaccgagaagtgagatgtccaagaattgtttgttaaaaccctcaaaactctaaggatattacctaataagtcgttggatgtaattcttatgatgatcacaaatcacacattcaatagttcaacgcaattaccgaaagtagcagggcgaaaatgtcattatcatgatcaaagggaccatgaatgtaattgcgcaatcagacgacgtgtcggtgaaaccaaaaaatttagaagaaaatatagctcactcaccaccactactcgaacctaacaccgcattaaatatcctacaccacttcctttctataaagaacacatcaacccacaaagcttttatctcgtttacatcgaaaccgctatatcattacatttacaacagcgctaaaaaaatatatataaagc

SEQ ID NO: 19 - DNA, 28 bp, Artificial Sequence
  primer TFNS1pBamHIRV
ggatccgctttatatatatttttttagc

SEQ ID NO: 20 - DNA, 1618 bp, Torenia Fournieri
  misc_feature: Trenia F3′5′H trancriptional region in pSPB3791
aatatttaaagcacaatctaaaacgaagcacagattcatttgtatgaagcacagattcatcaggggtattttagacaaaaacccgacgaatatcatccgttagatttattttgagcaaatccaatggcccattacttttgtccgcacatacggagaaaagtaccgtccgcatttgaaccagaggggatgggagggactgcgaacataatataattcgggtcaatggcgtaccaaaaaatggtcaaataaataatgctacaagttaaatgcacgtgattctcttgttgaccgttaaatgttgatttctattttgatcgaattttatagatattagctcagctgctgagactgagagagagattattttagaaacggatctgatcaagatcgctcgtgattattctcatcatctcgtgtagcatttagatttggtaaaaccgtaaaaatatcttgtcttaacttcttcctattaattccacaatctcacgcaaaaatgtatttttacagcgacatgccttgaggaggaatttcagtgctaaagacaaaatagacttagaaatgtgccgtttgattaattttggtagttaaaaaaaaaaaaattatttatttatttatgcttagccataataattaggacggaaacaagaagaatttgcccatttttaaggcctacgaatagtatacgggccataactttatgggggacattttcaggcctacccaatatgattgaatgtgcctccactccaattatcaaaaagaaacgccgactgagcttgagcacgattttcaaatgtctactccacagtgcaaaaattgcacgatcaattctgatgaaatgtattcccatctatgtagatgaatgggagcgtttgggatccaacactcgttttttctacctatttcgacggttttctctggtcaggtggtttccgagaggggactgaaatatacagtacagttgttgagatattgatgagatcaatcaagtcgtgtaagcttatctttctgaaaatataattatatttaattgattatcctaccgcttgtcaattttttctattgcgagtgtgttttggtgtaaagagtagctatcgatgctgaagagatttttatcaagtaagatgatgtggagaactaactgattgtttgttttgttgccagcgatatattgatgatgatgttctttgcaattagagttattctacttaacaaactttttctcttgtcataacatacacatagatactgcatgcttttccatgattcattctatctaataaaggtcaatctactccccactttcatccatgccaatgaaacacatacataaatggaaacatatcataatttaggtgtggccctattagttttcttctcgttgaacgtcggttgatggcctaaaaaaacgtgacaaaccacacagttgtcacccaacgtactccattgatacacatataaagctcagtgattgattattcataaacactcaactggttgagcactatttgcctcttctttgctagattcacatcgagtaagtactccgtccgaccttttattgtatggtataatcgtaagaaaacaaattaagcagctagtcaccattcagtctaga

SEQ ID NO: 21 - DNA, 1416 bp, Torenia Fournieri
  misc_feature: Trenia FNS transcriptional region in pSPB3789
ctagtagataatatcgtgaatgatatttgtaaagaacgttgtattaaattcaggacacttaattgatgaatcacaatcaaataatattattttgcagtatctggtacataagtatgatataaatgataagtgccataactgtgagaatataaactataatcgtaataaaaatttgtatagcataaaatatttaatatttactataaattcaataaaatcaatgctacatgattatgtaagatgatgtggattttcttaacaaaatgattttggcttgatcgtgatgatactattaaaaaataatgaagtttgtctaggacatggcattggtttatagatagatgtcctcatacgtttattggcacttgacatttgtttctttttattcttgtgaattgggatatgtggatttataaatatttttagaagtctggcttgtatttctgatactaatttttatgatataatttctttatgaattaattgatattaaaggctcatttgaagtttgcggatgattcaatgcgagcaatagcgtatatattgtgattggttggtgcactgcacatataggctcgcatcgaatcatccgccgtttgcttaggaaattataagactcattttattaatttaaaaatagaatttgggtgaaaggtagacgtacggtacggctacggtacctaaccttcgaggcttagactaagggtaaagaaaagaccaggaaaagaaaaaatctagaatcgttaaaatattaaatgcaattaaatgaaaataaaaaaggataaataaagatccacaccacacctatcaattgtggagtctaattgtttgaacttgatacaatacaatgatatctgaagtatcgtatcgaaatacacacgttatgtttttcctatttggcacatcatatttattcttggtttttcaaatccaagatacaaaaactcataaaatacctcagtcaccaaaaataacactaacagatttcaaacatactgcatgattatttaaaattaaaaccgagaagtgagatgtccaagaattgtttgttaaaaccctcaaaactctaaggatattacctaataagtcgttggatgtaattcttatgatgatcacaaatcacacattcaatagttcaacgcaattaccgaaagtagcagggcgaaaatgtcattatcatgatcaaagggaccatgaatgtaattgcgcaatcagacgacgtgtcggtgaaaccaaaaaatttagaagaaaatatagctcactcaccaccactactcgaacctaacaccgcattaaatatcctacaccacttcctttctataaagaacacatcaacccacaaagcttttatctcgtttacatcgaaaccgctatatcattacatttacaacagcgctaaaaaaatatatataaagc